(19) United States
(12) Patent Application Publication
(10) Pub. No.: US 2012/0265164 A1
(43) Pub. Date: Oct. 18, 2012

(54) FLOW RESTRICTOR FOR MEDICAL DEVICES

(75) Inventors: Markus W. Reiterer, Plymouth, MN (US); Dale F. Seeley, Spring Park, MN (US); Kimberly A. Chaffin, Woodbury, MN (US)

(73) Assignee: MEDTRONIC, INC., Minneapolis, MN (US)

(21) Appl. No.: 13/444,330

(22) Filed: Apr. 11, 2012

Related U.S. Application Data
(60) Provisional application No. 61/475,254, filed on Apr. 14, 2011.

(51) Int. Cl. A61M 25/14; C08J 3/28; B82Y 30/00

(52) U.S. Cl. 604/500; 604/246; 522/2; 977/773

(57) ABSTRACT

This relates to fluid delivery devices and methods and, more particularly, to implantable fluid delivery devices and methods of use. A flow restrictor for medical devices is described with particularly useful properties.

This application claims the benefit of U.S. Provisional Patent Application Ser. No. 61/475,254 by Reiterer et al., which was filed Apr. 14, 2011, and is entitled “FLOW RESTRICTOR FOR MEDICAL DEVICES.” U.S. Provisional Patent Application Ser. No. 61/475,254 is incorporated herein by reference in its entirety.

### TECHNICAL FIELD

This disclosure relates to fluid delivery devices and, more particularly, to implantable fluid delivery devices.

### BACKGROUND

Fluid delivery devices are used to treat a number of physiological, psychological, and emotional conditions, including chronic pain, movement disorders, tremor, Parkinson's disease, epilepsy, urinary or fecal incontinence, diabetes, sexual dysfunction, obesity, spasticity, or gastroparesis. For some medical conditions, a fluid delivery device provides more effective therapy compared to other therapy options.

A fluid delivery device may provide a patient with a fixed or programmable dosage or infusion of a drug or other therapeutic agent. The fluid delivery device typically includes a reservoir for storing the therapeutic agent, a fill port, a mechanism to pump and meter the therapeutic agent from the reservoir, a catheter port to transport the therapeutic agent from the reservoir to a therapy site via a catheter, and electronics to control the pumping mechanism.

### SUMMARY

In general, the present disclosure is directed to a flow restrictor for controlling the flow rate of a therapeutic fluid that is delivered to a target tissue within a patient. The flow restrictor may be made by a multi-photon polymerization (MPP) process, such as two-photon polymerization (2PP), which allows the flow restrictor to comprise a fluid path sized to be small enough to provide relatively small volumetric flow rates of the therapeutic fluid while still providing a small flow restrictor that may be implantable within a patient.

In one example, the present disclosure is directed to a method comprising applying a multi-photon polymerization (MPP) process to a material to define a fluid flow restrictor for a medical device. In one example, applying the multi-photon polymerization process comprises forming a body comprising a first end, a second end, a fluid inlet proximate the first end, a fluid outlet, and a fluid path between the fluid inlet and the fluid outlet, wherein forming at least a portion of the body comprises, selecting a location for each of a plurality of focal volumes within a resin, the resin comprising a monomer and a photoinitiator sensitive to light having a wavelength range, wherein the photoinitiator is configured to initiate polymerization of the monomer within one of the plurality of focal volumes when two or more photons of light having the wavelength range are absorbed by the photoinitiator within the one of the plurality of focal volumes, wherein the plurality of selected focal volumes form a shape of a portion of a body defining the fluid flow restrictor, and sequentially focusing a laser into each of the plurality of selected focal volumes within the resin to polymerize the monomer and form the portion of the body, wherein the laser is configured to provide for multi-photon absorption at the wavelength range within each of the plurality of selected focal volumes.

In another example, the present disclosure is directed to a flow restrictor for a medical device, the flow restrictor comprising a body having a first end, a second end, a fluid inlet proximate the first end, a fluid outlet, and a fluid path between the fluid inlet and the fluid outlet, wherein the body is made from a photocrosslinkable polymer that is formed by multi-photon polymerization of the polymer.

In yet another example, the present disclosure is directed to a system comprising a fluid delivery device, a catheter comprising an proximal end coupled to the fluid delivery device, a distal end implantable proximate a target tissue, and a lumen extending from the proximal end to the distal end, a flow restrictor coupled to the distal end of the catheter, the flow restrictor comprising a body having a first end, a second end, a fluid inlet proximate the first end in fluid communication with the catheter lumen, a fluid outlet, and a fluid path between the fluid inlet and the fluid outlet, wherein the flow restrictor body is made from a photocrosslinkable polymer that is formed by multi-photon polymerization of the polymer.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example therapy system that includes an implantable fluid delivery device for the delivery of a therapeutic fluid to a target tissue of a patient.

FIG. 2 is a conceptual diagram illustrating another example therapy system that includes an implantable fluid delivery device for the delivery of therapeutic fluid.

FIG. 3 is a block diagram illustrating various example components of an example implantable fluid delivery device.

FIG. 4 is a conceptual diagram showing an osmotic pump for the delivery of a therapeutic fluid.

FIG. 5 is a perspective view of an example flow restrictor comprising a generally helical fluid path that may be used for the delivery of a therapeutic fluid to a patient.

FIG. 6 is a cross-sectional view of an example flow restrictor comprising a generally serpentine fluid path that may be used for the delivery of a therapeutic fluid to a patient.

FIG. 7 is a perspective view of an example flow restrictor comprising two generally helical fluid paths that may be used for the delivery of one or more therapeutic fluids to a patient.

FIG. 8 is a perspective view of an example flow restrictor comprising two generally helical fluid paths that join together at a junction portion in order to mix a therapeutic fluid from the first fluid path with a therapeutic fluid from the second fluid path.

FIG. 9 is a perspective view of an example flow restrictor comprising a common feed portion that splits into two generally helical fluid paths in order to deliver a therapeutic fluid to more than one fluid outlet.

FIG. 10 is a conceptual diagram of a two-dimensional plane comprising a plurality of voxels that have been formed by multi-photon polymerization.

FIG. 11 is a flow diagram of an example method of forming a flow restrictor that may be used for the delivery of a therapeutic fluid to a patient.

### DETAILED DESCRIPTION

In general, the present disclosure is directed to a flow restrictor for controlling the flow rate of a therapeutic fluid that is delivered to a target tissue within a patient. The flow restrictor may be made by a multi-photon polymerization process, such as two-photon polymerization, which allows the flow restrictor to comprise a fluid path sized to be small enough to provide for relatively small volumetric flow rates of the therapeutic fluid, such as between about 0.25 microliters per hour and about 50 microliters per hour, while still providing a small flow restrictor that may be implantable within a patient. In some examples, the fluid path may have a width, such as a diameter of a generally circular cross-sectioned fluid path, of between about 1 micrometers and about 20 micrometers. Multi-photon polymerization also allows the fluid path of the flow restrictor to have a fully three-dimensional shape, such as a generally helical or serpentine fluid path, in order to provide a desired length of the fluid path in order to adjust the resulting flow rate that is passed through flow restrictor.

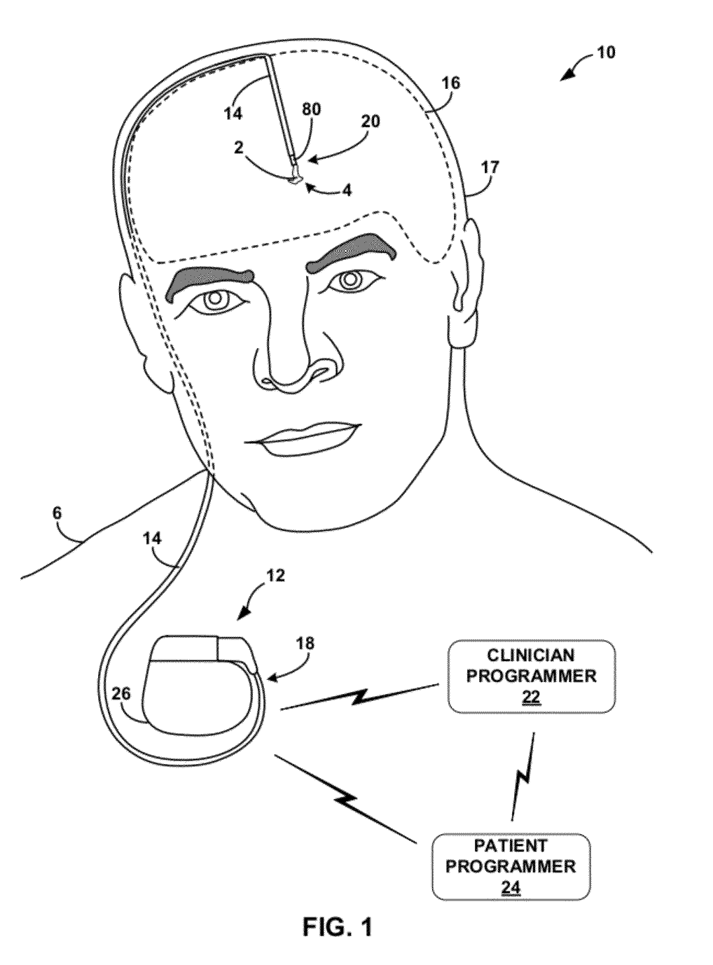

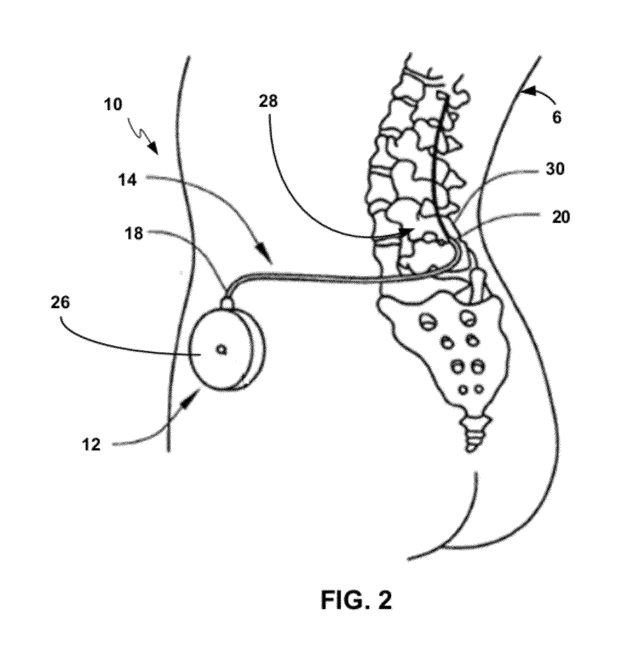

FIGS. 1 and 2 are conceptual diagrams illustrating an example system 10 for the delivery of a therapeutic fluid 2 from an implantable medical device (IMD) 12 to a target site 4, 28 within a patient 6. The therapeutic fluid 2 may comprise a pharmaceutical agent such as, for example, a drug, insulin, pain relieving agent, anti-inflammatory agent, gene therapy agent, or the like, that produces a therapeutic effect on patient 6 when delivered to target site 4, 28. IMD 12 delivers the therapeutic fluid to target site 4 through one or more catheters 14 coupled to IMD 12. The catheter may comprise a plurality of catheter segments or the catheter may be a unitary catheter. In the example shown in FIG. 1, target site 4 is within the brain 16 of patient 6. In the example shown in FIG. 2, a target site 28 is within the spinal cord 30 of patient 6. An IMD in accordance with the present disclosure may be used to deliver a therapeutic fluid to other target sites within a patient 6, such as proximate or within an internal organ, such as the liver or the pancreas.

A proximal end 18 of catheter 14 is coupled to IMD 12 while a distal end 20 is located proximate to target site 4, 28. Stereotactic techniques or other positioning techniques may be used to precisely position fluid delivery catheter 14 with respect to target site 4, 28 and to maintain the precise positioning throughout use. In some examples, after positioning, one or more fluid delivery catheters 14 may be held precisely in place using fixation techniques or mechanisms such as those similar to the Medtronic StimLoc™ burr hole cover, manufactured by Medtronic, Inc., of Minneapolis, Minn.

System 10 also may include a clinician programmer 22 and/or a patient programmer 24. Clinician programmer 22 may be a handheld computing device that comprises a user interface, such as a display viewable by a user and a user input mechanism that may be used by the user to provide input to clinician programmer 22. In one example, the user interface of clinician programmer 22 may comprise a keypad, buttons, a peripheral pointing device, touch screen, voice recognition, or another input mechanism that allows the user to navigate though the user interface of programmer 22 and provide input.

Clinician programmer 22 may permit a clinician to program therapy for patient 6 via the user interface. For example, using clinician programmer 22, the clinician may specify fluid delivery parameters, i.e., create programs, for use in delivery of therapy. In another example, clinician programmer 22 may be used to transmit initial programming information to IMD 12. This initial information may include hardware information for system 10 such as the type of catheter 14, the position of catheter 14 within patient 6, the type of therapeutic fluid(s) delivered by IMD 12, a baseline orientation of at least a portion of IMD 12 relative to a reference point, therapy parameters of therapy programs stored within IMD 12 or within clinician programmer 22, and any other information the clinician desires to program into IMD 12.

During a programming session, the clinician may determine one or more therapy programs, which may include one or more therapy schedules, programmed doses, dose rates of the programmed doses, and specific times to deliver the programmed doses that may provide effective therapy to patient 6. In one example, programmer 22 may be configured to program an output pressure that is provided by a pumping mechanism, which in turn may modify a flow rate through a flow restrictor, as described in more detail below. In another example, a clinician, using programmer 22, may select a desired output flow rate to patient 6, and programmer 22 or IMD 12 may calculate the necessary output pressure from a pumping mechanism to achieve the selected flow rate. Patient 6 may provide feedback to the clinician as to the efficacy of a specific therapy program being evaluated or desired modifications to the therapy program. Once the clinician has identified one or more programs that may be beneficial to patient 6, patient 6 may continue the evaluation process using patient programmer 24 and determine which dosing program or therapy schedule best alleviates the condition of patient 6 or otherwise provides efficacious therapy to patient 6.

Clinician programmer 22 may support telemetry (e.g., radio frequency (RF) telemetry) with IMD 12 to download programs and, optionally, upload operational or physiological data stored by IMD 12. In this manner, the clinician may periodically interrogate IMD 12 to evaluate efficacy and, if necessary, modify the programs or create new programs. In some examples, clinician programmer 22 transmits programs to patient programmer 24 in addition to or instead of IMD 12.

Like clinician programmer 22, patient programmer 24 may be a handheld computing device. Patient programmer 24 may also include a user interface to allow patient 6 to interact with patient programmer 24 and IMD 12. In this manner, patient programmer 24 provides patient 6 with a user interface for control of the fluid delivery therapy delivered by IMD 12. For example, patient 6 may use patient programmer 24 to start, stop or adjust the therapy provided by IMD. In particular, patient programmer 24 may permit patient 6 to select adjust parameters of a program such as duration of treatment, frequency of treatment, and the like. Patient 6 may also select a program, e.g., from among a plurality of stored programs, as the present program to control delivery of fluid by IMD 12.

In some examples, patient programmer 24 may have limited functionality in order to prevent patient 6 from altering critical functions or applications that may be detrimental to patient 6. In this manner, patient programmer 24 may only allow patient 6 to adjust certain therapy parameters or set an available range for a particular therapy parameter. In some cases, a patient programmer 24 may permit patient 6 to control IMD 12 to deliver a supplemental, patient bolus, if permitted by the applicable therapy program administered by the IMD 12, e.g., if delivery of a patient bolus would not violate a lockout interval or maximum dosage limit. Patient programmer 24 may also provide an indication to patient 6 when therapy is being delivered or when IMD 12 needs to be refilled or when the power source within patient programmer 24 or IMD 12 needs to be replaced or recharged.

In other examples, rather than being a handheld computing device or a dedicated computing device, either or both of clinician programmer 22 or patient programmer 24 may be a larger workstation or a separate application within another multi-function device. For example, the multi-function device may be a cellular phone, personal computer, laptop, workstation computer, or personal digital assistant that can be configured to simulate programmer 22, 24. Alternatively, a notebook computer, tablet computer, or other personal computer may execute an application to function as programmer 22, 24, e.g., with a wireless adapter connected to the personal computer for communicating with IMD 12.

IMD 12, clinician programmer 22, and patient programmer 24 may communicate via cables or via a wireless communication, as shown in FIG. 1. Clinician programmer 22 and patient programmer 24 may, for example, communicate via wireless communication with IMD 12 using radio frequency (RF) telemetry techniques known in the art. Clinician programmer 22 and patient programmer 24 also may communicate with each other using any of a variety of RF, infrared or other communication techniques. Each of clinician programmer 22 and patient programmer 24 may include a transceiver to permit bi-directional communication with IMD 12. Each programmer 22, 24 may also communicate with another programmer or computing device via exchange of removable media, such as magnetic or optical disks, or memory cards or sticks. Further, each programmer 22, 24 may communicate with IMD 12 and another programmer via remote telemetry techniques, communicating via a local area network (LAN), wide area network (WAN), public switched telephone network (PSTN), or cellular telephone network, for example.

Although patient 6 is generally referred to as a human patient in the present disclosure, system 10 can be used with other mammalian or non-mammalian patients. IMD 12 may be employed to treat, manage or otherwise control various conditions or disorders of patient 6, including, e.g., pain (e.g., chronic pain, post-operative pain or peripheral and localized pain), tremor, movement disorders (e.g., Parkinson's disease), diabetes, epilepsy, neuralgia, chronic migraines, urinary or fecal incontinence, sexual dysfunction, obesity, gastroparesis, eye disorders, kidney disorders, liver disorders, pancreatic disorders, mood disorders, dementia (e.g. Alzheimer's disease) or other disorders.

IMD 12 may be configured to deliver one or more therapeutic fluids, alone or in combination with other therapies, including, e.g., electrical or optical stimulation. For example, in some cases, a medical pump may deliver one or more pain-relieving drugs to patients with chronic pain, insulin to a patient with diabetes, or other fluids to patients with different disorders. IMD 12 may be implanted in patient 6 for chronic or temporary therapy delivery.

IMD 12 includes an outer housing 26 that is constructed of a biocompatible material that resists corrosion and degradation from bodily fluids, such as titanium or biologically inert polymers. IMD 12 may be implanted within a subcutaneous pocket close to target site 4, or as close to the target site 4 as is practical. For example, as shown in FIG. 1 wherein target site 4 is within the brain 16, IMD 12 may be implanted within a subcutaneous pocket in a clavicle region of patient 6. In other examples, IMD 12 may be implanted within other suitable sites within patient 6, which may depend, for example, on where the target site is located within patient 6, and the ease of implanting IMD 12 within suitable locations near the target site. For example, as shown in FIG. 2, if a target site 28 is proximate the spinal cord 30 of patient 6, then IMD 12 may be implanted within the abdomen of patient 6 close to the position along spinal cord 30 where target site 28 is located.

Catheter 14 may be implanted using a stylet for insertion stiffness while the catheter 14 is being implanted in patent 6. For example, the stylet may allow a surgeon to easily manipulate catheter 14 as it is guided from the clavical region, though the neck, into cranium 17, and into brain 16 of patient 6. The stylet may be removable after insertion of catheter 14 so that catheter 14 is flexible after insertion such that the stylet does not interfere with chronic treatment by catheter 14. In one example, catheter 14 may include a stylet lumen for receiving the stylet and for allow the removal of the stylet.

Catheter 14 may be coupled to IMD 12 either directly or with the aid of a catheter extension (not shown). In the example shown in FIG. 1, catheter 14 traverses from the implant site of IMD 12 to target site 4 within brain 16. Catheter 14 is positioned such that one or more fluid delivery outlets of catheter 14 are proximate to one or more locations within patient 6. In the example shown in FIG. 1, IMD 12 delivers a therapeutic fluid 2 to one or more locations at target site 4 within patient 6. IMD 12 delivers a therapeutic fluid to target site 4 within brain 16 with the aid of catheter 14.

In some examples, multiple catheters may be coupled to IMD 12 to target the same or different tissue or nerve sites within patient 6. Thus, in some examples, system 10 may include multiple catheters or catheter 14 may define multiple lumens for delivering different therapeutic agents to patient 6 or for delivering a therapeutic fluid to different tissue sites within patient 6. Accordingly, in some examples, IMD 12 may include a plurality of reservoirs for storing more than one type of therapeutic fluid. In some examples, IMD 12 may include a single long tube that contains the therapeutic agent in place of a reservoir. However, for ease of description, an IMD 12 including a single reservoir is primarily discussed herein with reference to the example of FIG. 1.

IMD 12 may deliver one or more therapeutic fluids 2 to patient 6 according to one or more therapy programs. Example therapeutic fluids that IMD 12 may be configured to deliver include insulin, baclofen, morphine, hydromorphone, bupivacaine, clonidine, other analgesics, genetic agents, antibiotics, nutritional fluids, analgesics, hormones or hormonal drugs, gene therapy drugs, proteins, cells, peptides, anticoagulants, cardiovascular medications or chemotherapeutics. A therapy program, generally speaking, may set forth different therapy parameters, such as a therapy schedule specifying programmed doses, dose rates for the programmed doses, and specific times to deliver the programmed doses.

The therapy programs may be a part of a program group for therapy, wherein the group includes a plurality of constituent therapy programs and/or therapy schedules. In some examples, IMD 12 may be configured to deliver a therapeutic agent to patient 6 according to different therapy programs on a selective basis. IMD 12 may include a memory to store one or more therapy programs, instructions defining the extent to which a clinician or patient 6 may adjust therapy parameters, switch between therapy programs, or undertake other therapy adjustments. A clinician may select and/or generate additional therapy programs for use by IMD 12 via clinician programmer 22. Patient 6 may select and/or generate additional therapy programs for use by IMD 12 via external programmer 24 at any time during therapy or as designated by the clinician.

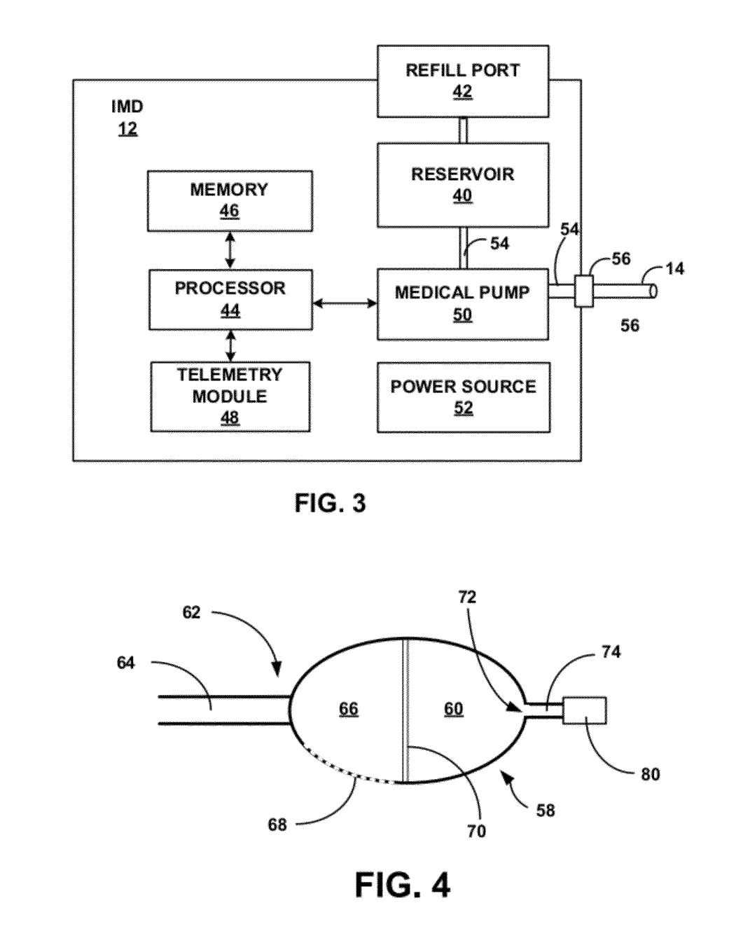

FIG. 3 is a functional block diagram illustrating components of an example of IMD 12. The example IMD 12 shown in FIG. 2 includes reservoir 40, refill port 42, processor 44, memory 46, telemetry module 48, medical pump 50, power source 52, internal channels 54, and catheter access port 56.

Refill port 42 may comprise a self-sealing injection port. The self-sealing injection port 42 may include a self-sealing membrane to prevent loss of therapeutic agent delivered to reservoir 40 via refill port 42. After a delivery system, e.g., a hypodermic needle, penetrates the membrane of refill port 42, the membrane may seal shut when the delivery system is removed from refill port 42. Internal channels 54 comprises one or more segments of tubing or a series of cavities that run from reservoir 40, around or through medical pump 50 to catheter access port 56.

Processor 44 may control the operation of medical pump 50 with the aid of software instructions associated with program information that is stored in memory 46. In one example, processor 44 is configured to run the software instructions in order to control operation of IMD 12. For example, the software instructions may define therapy programs that specify the amount of a therapeutic agent that is delivered to a target tissue site within patient 6 from reservoir 40 via catheter 14, e.g., dose, the rate at which the agent is delivered, e.g., dosage rate, and the time at which the agent will be delivered and the time interval over which the agent will be delivered, e.g., the therapy schedule for dose or doses defined by program. In other examples, a quantity of the therapeutic agent may be delivered according to one or more physiological characteristics of a patient, e.g., physiological characteristics sensed by one or more sensors (not shown) implanted within a patient as part of therapy system 10 (FIG. 1).

Processor 44 can include one or more processors, including one or more microprocessors, digital signal processors (DSPs), application specific integrated circuits (ASICs), field programmable gate arrays (FPGAs), or any other equivalent integrated or discrete logic circuitry, as well as any suitable combination of such components. The term “processor” or “processing circuitry” may generally refer to any of the foregoing logic circuitry, alone or in combination with other logic circuitry, or any other equivalent circuitry.

Memory 46 may include any volatile or non-volatile media, such as a random access memory (RAM), read only memory (ROM), non-volatile RAM (NVRAM), electrically erasable programmable ROM (EEPROM), flash memory, and the like. As mentioned above, memory 46 may store program information including instructions for execution by processor 44, such as, but not limited to, therapy programs, historical therapy programs, timing programs for delivery of the therapeutic agent from reservoir 40 to catheter 14, and any other information regarding therapy of patient 6. Memory 46 may include separate memory portions for storing instructions, patient information, therapy parameters (e.g., grouped into sets referred to as “dosing programs”), therapy adjustment information, program histories, and other categories of information such as any other data that may benefit from separate physical memory modules.

Telemetry module 48 in IMD 12, as well as telemetry modules in programmers, such as external programmer 20, may accomplish communication by RF communication techniques. In addition, telemetry module 48 may communicate with clinician programmer 22 and/or patient programmer 24 via proximal inductive interaction of IMD 12 with programmer 22, 24. Processor 44 may control telemetry module 48 to send and receive information.

Power source 52 delivers operating power to various components of IMD 12. Power source 52 may include a small rechargeable or non-rechargeable battery and a power generation circuit to produce the operating power. Medical pump 50 may be a mechanism that delivers a therapeutic agent in some metered or other desired flow dosage to target site 2 within patient 6 from reservoir 40 via catheter 14. Medical pump 50 may comprise an active pumping mechanism or a passive pumping mechanism. An active pumping mechanism may comprise an actuation mechanism, such as a piston, that is electrically actuated to provide a pump stroke to move fluid from reservoir 40. For example the actuation mechanism may comprise an electromagnetic coil and an actuator that is movable in response to electrical energization of the coil. Other actuation mechanisms may be used, such as a piezoactuator. An example of an active medical pump is the SYNCHROMED II medical pump manufactured by Medtronic, Inc., Minneapolis, Minn.

A passive pumping mechanism may comprise a pressurized reservoir wherein the pressure in the reservoir acts to deliver the therapeutic fluid from the reservoir. An example of a passive pumping device is the ISOMED medical pump manufactured by Medtronic, Inc., Minneapolis, Minn. Another example of a passive pumping device is described in U.S. Publication No. 2007/0043335, published on Feb. 22, 2007, the entire disclosure of which is incorporated herein by reference as if reproduced in its entirety. Another example of a passive pumping mechanism is an osmotically-driven pump 58 that includes a fluid reservoir 60, as shown in FIG. 4. As shown schematically in FIG. 4, pump 58 and reservoir 60 may be located proximate a distal end 62 of a delivery device, such as a catheter (e.g., catheter 14) or a lead 64, rather than within an IMD. In one example, lead 64 and pump 58 are implantable proximate the target site 4 so that therapeutic fluid 2 may be delivered to target site 4. In one example, pump 58 comprises a small fluid reservoir 60 containing the therapeutic fluid 2 that is to be delivered to the target tissue. Pump 58 may be an osmotic pump that utilizes the principles of osmosis to force fluid from reservoir 60.

Osmosis is the transfer of a solvent, e.g., water, across a barrier, generally from an area of lesser solute concentration to an area of greater solute concentration. In one example, osmotic pump 58 may be adapted to cause fluid to flow from the patient's surrounding tissue into a small compartment 66 through a semi-permeable membrane 68. This ingress of fluid into compartment 66, in turn, displaces a barrier 70 located between compartment 66 and the adjacent reservoir 60 containing the therapeutic fluid. Displacement of barrier 70 forces the therapeutic fluid from reservoir 60 into the patient's body at a controlled rate, for example through an opening 72 in reservoir 60 and/or through a delivery outlet tube 74.

Delivery may occur after reservoir 60 is immersed in the body fluid. The rate of delivery may be modified, for example, by selection of dimensions of compartment 66 and fluid reservoir 60, the flexibility and dimension of displaceable barrier 70, the size of opening 72 from fluid reservoir 60, the construction of permeable membrane 68, and/or the environment within compartment 66 into which the body fluid flows. Descriptions of osmotic pumps may be found in commonly assigned U.S. Patent Application Publication Nos. 2009/0281528 and 2008/0102119 entitled “Osmotic Pump Apparatus and Associated Methods,” both of which are incorporated herein by reference in their entirety.

Controlling the flow rate from passive pumping devices can be more difficult than from active pumping devices. However, system 10 includes a flow restrictor 80 located within the flow path of therapeutic agent 2, e.g. at distal end 20 of catheter 14 (FIG. 1) or on outlet tube 74 of osmotic pump 58 (FIG. 4). A flow restrictor 80 may be located at other places along the flow path of therapeutic agent 2, such as within catheter 14 or within IMD 12, for example at an outlet of a pumping mechanism 50 within IMD 12.

Flow restrictor 80 is configured to restrict the volume of therapeutic fluid 2 that can exit from catheter 14. Therefore, flow restrictor 80 provides for a controlled flow rate of therapeutic fluid 2 from catheter 14 so that a passive pumping mechanism can be used within IMD 12. Because passive pumping mechanisms tend to be smaller and cheaper than active pumping mechanisms, flow restrictor 80 allows for a smaller and less expensive IMD 12 while still allowing for a selected flow rate of therapeutic fluid 2 to be delivered to target site 4. While a passive pumping mechanism may be used, IMD 12 is not so limited. Rather, an active pumping mechanism, such as a piston, may still be used along with flow restrictor 80 in order to provide variable control over the back pressure of fluid being fed into flow restrictor 80, which provides for some variability in the rate of fluid that is delivered to target site 4. In another example, a flow restrictor may be used with an active pumping mechanism to provide a maximum flow rate that may be delivered to a patient as a safety redundancy measure in the event that the active pumping mechanism malfunctions and delivers a larger flow rate than is intended for the patient. In such an example, the flow restrictor may be configured to restrict the flow rate that is delivered to a target site to a maximum default flow rate. In other examples, a flow restrictor in accordance with the present disclosure may be used to control the flow rate through a shunt for the removal or drainage of excess fluid from a tissue within patient.

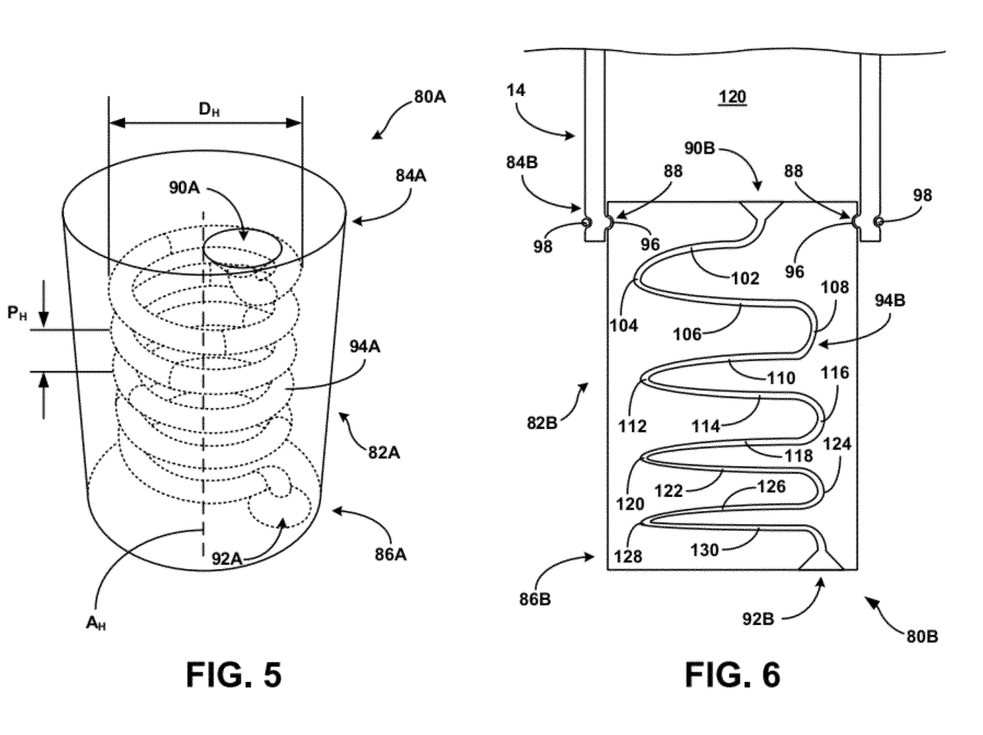

FIGS. 5-9 show close-up views of example flow restrictors 80A, 80B, 80C, 80D, and 80E (referred to collectively herein as “flow restrictor 80”) that may be used to control the flow of therapeutic fluid 2 from catheter 14. Flow restrictor 80 may also be used to control the flow of therapeutic fluid 2 from outlet tube 74 of osmotic pump 58, as shown in FIG. 4. However, for the sake of brevity, flow restrictor 80 will be described with respect to its use with catheter 14. In one example, flow restrictor 80 comprises a body 82A, 82B, 82C, 82D, 82E (referred to collectively herein as “body 82”) having, respectively, a first end 84A, 84B, 84C, 84D, 84E (referred to collectively herein as “first end 84”), and a second end 86A, 86B, 86C, 86D, 86E (referred to collectively herein as “second end 86”). Flow restrictor 80 may also comprise an attachment structure 88 configured for attaching body 82 to catheter 14 (FIG. 6). Body 82 also includes a fluid inlet 90A, 90B, 90C, 90D, 90E (referred to collectively herein as “fluid inlet 90”) proximate first end 84, a fluid outlet 92A, 92B, 92C, 92D, 92E (referred to collectively herein as “fluid outlet”), and at least one fluid path 94A, 94B, 94C, 94D, 94E (referred to collectively herein as “fluid path 94”) between fluid inlet 90 and fluid outlet 92.

In one example, an attachment structure 88 is located proximate first end 84 so that catheter 14 is attached to flow restrictor 80 proximate to fluid inlet 90. Attachment structure 88 may take many forms capable of providing a sealing connection between flow restrictor 80 and catheter 14. In the example shown in FIG. 6, attachment structure 88 comprises a groove 96 within body 82B of flow restrictor 80B capable of receiving a resilient portion of catheter 14. In one example, the resilient portion of catheter 14 comprises a resilient ring 98, such as a resilient metal ring 98, which fits over catheter 14 and flow restrictor 80. In one example, metal ring 98 exerts a force radially inwardly onto catheter 14 so that a portion of catheter 14 is forced into groove 96. In another example, catheter 14 may comprise one or more detents (not shown) that are received by groove 96. In another example, the flow restrictor may comprise a detent extending radially outwardly from the flow restrictor body that is received by a groove within an interior surface of a catheter (not shown). Other attachment structures 88 may be possible, such as mated threading on flow restrictor 80 and the distal end of catheter 14, structures that provide an interference fit or snap fit between flow restrictor 80 and catheter 14, such as a taper on an outer surface of flow restrictor 80 and a corresponding mating taper on an interior surface of catheter 14, one or more fasteners, adhesive bonding, primer/adhesive bonding, and welding between flow restrictor 80 and catheter 14, such as solvent welding, thermal welding, or sonic welding.

Fluid path 94 extends between fluid inlet 90 and fluid outlet 92 of flow restrictor 80. Because fluid path 94 has a relatively small inner diameter compared to the inner diameter of the fluid lumen within catheter 14, fluid path 94 acts to limit the flow rate of therapeutic fluid 2 that can flow through flow restrictor 80. Flow restrictor 80 is designed so that fluid path 94 provides a desired flow rate for a particular therapeutic fluid 2. The flow rate of therapeutic fluid 2 that may flow through flow restrictor 80 depends on several parameters, but the most prominent parameters include the applied back pressure P, and more particularly the change in pressure (ΔP) between fluid inlet 90 and fluid outlet 92, the length L_{P} of fluid path 94, the inner width W_{P} of fluid path 94 (which, if fluid path 94 has a generally circular cross-section, is the inner diameter D_{P} of fluid path), the surface energy of the interior surface of fluid path 94, and the viscosity μ of therapeutic fluid 2. The length L_{P} and inner width W_{P} of fluid path 94 are of particular interest, because the length L_{P} and inner width W_{P} are controllable by modifying the physical shape of fluid path 94 within flow restrictor 80. In general, the flow rate Q of therapeutic fluid 2 is proportional to the length L_{P} of fluid path 94 over the width W_{P} of fluid path 94 to the power of four, as shown in Equation 1:

$$Q ∝ \frac{{W}_{P}^{4}}{{L}_{P}} \qquad [ 1 ]$$

Therefore, in some examples, fluid path 94 is configured to provide a length L_{P} and width W_{P} that may provide for a desired flow rate Q of therapeutic fluid 2. In some examples, the desired flow rate Q of therapeutic fluid 2 is between about 0.25 microliters per hour and about 50 microliters per hour, for example between 10 microliters and about 30 microliters. In another example, fluid path 94 may be configured to provide for a small flow rate of between about 1 microliter per hour and about 5 microliters per hour, such as about 2 microliters per hour. As described in more detail below, in order to provide for a flow rate on the order of only a few microliters per hour, the width W_{P} of fluid path 94 may need to be particularly small, e.g., on the order of about 1 micrometers to about 20 micrometers, while the length L_{P} of fluid path 94 may need to be on the order of between about 0.5 millimeters and about 50 millimeters, such as about 3 millimeters. In some examples, fluid path 94 may be designed to provide for a change in pressure (ΔP) of between about 50 kilopascals (about 7.25 pounds per square inch (PSI)) and about 400 kilopascals (about 58 PSI), such as between about 135 kilopascals (about 19.5 PSI) and about 250 kilopascals (about 36.3 PSI).

In some examples, the relatively small size of fluid path 94, e.g., with a width of between about 1 micrometers and about 20 micrometers, allows for a relatively small volumetric flow rate, e.g., between about 0.25 microliters per hour and about 50 microliters per hour, while still providing for a relatively high fluid velocity exiting fluid outlet 92 of flow restrictor 80. In one example, the small cross-sectional area of fluid path 94 allows therapeutic fluid 2 to pass through flow restrictor 80 at a local fluid velocity of between about 1 millimeter per second and about 20 millimeters per second. Such flow rates may allow for reduced or eliminated tissue ingrowth into fluid path 94, such as via tissue intima buildup around flow restrictor 80 or cell migration into fluid path 94, and thus may reduce or eliminate occlusion of fluid path 94 after implantation.

In some examples, it is desirable for the overall dimensions of body 82 of flow restrictor 80 to be as small as is practical, so that the total length L_{R} and lateral width W_{R} of flow restrictor 80 are each substantially shorter than a desired length L_{P} of fluid path 94. For example, when flow restrictor 80 is to be implantable within brain 16 of patient 6 (FIG. 1) or within the spinal cord 30 (FIG. 2), flow restrictor 80 may be sized to have an outer width (e.g., an outer diameter) of between about 0.5 millimeters and about 3 millimeters, such as between about 0.6 millimeters and about 1.3 millimeters. Therefore, in some examples, fluid path 94 may have a geometry that provides for a tortuous or winding fluid path 94 in order to fit a fluid path 94 having a length L_{P} that is larger than the overall length L_{R} of flow restrictor 80. FIGS. 5-9 show examples of fluid paths 94 that may be used to accommodate the desired length L. As will be understood by a person of ordinary skill in the art, the fluid paths 94 shown in FIGS. 5-9 are not meant to be limiting or exhaustive. Other fluid path geometries could be contemplated without varying from the scope of the present disclosure.

FIG. 5 shows an example flow restrictor 80A having a generally cylindrical helical fluid path 94A. Helical fluid path 94A comprises a generally helically wound fluid path starting at fluid inlet 92A and extending to fluid outlet 94A. Helical fluid path 94A has a helical diameter D_{H} defined as twice the distance from a central axis A_{H} to an outermost extent of fluid path 94A, and a helical pitch length P_{H}, defined as the distance of one complete helical turn measured parallel to axis A_{H}. The helical diameter D_{H}, pitch P_{H}, and the number of helical turns may be selected so that a desired length of fluid path 94 is achieved.

In another example, not shown, a generally helical fluid path may be generally conically helical in shape so that the helical diameter starts out relatively small at fluid inlet 90A and increases between fluid inlet 90A and fluid outlet 92A or starts out relative large at fluid inlet 90A and decreases between fluid inlet 90A and fluid outlet 92A. In other examples, not shown, the helical diameter may increase for a portion of fluid path 94A remain constant for a portion of fluid path 94, and/or decrease for another portion of fluid path 94A. In yet other examples, the pitch P_{H} may change along the length of fluid path 94A, for example by increasing or decreasing as fluid path 94A gets farther and farther from fluid inlet 90A. Other combinations of varying pitch or helical diameter are also possible.

FIG. 6 shows a lateral cross section of an example flow restrictor 80B having a fluid path 94B having a generally serpentine (e.g., back and forth or “meandering”) shape. In one example, shown in FIG. 6, a first section 102 of serpentine fluid path 94B extends generally from fluid inlet 90B proximate first end 84B generally laterally (i.e., in a direction substantially normal to a longitudinal extent of flow restrictor 80B) across body 82B then undergoes a turn 104 so that a second section 106 of fluid path 94B extends laterally back across body 82B in generally the opposite direction of first section 102. Fluid path 94B may undergo additional turns, such as turns 108, 112, 116, 120, 124, and 128 in order to form additional sections 110, 114, 118, 122, and 130 of fluid path 94B. The length of fluid path 94B may be achieved by selecting the number of sections 102, 106, 110, 114, 118, 122, 130 and the length of each section 102, 106, 110, 114, 118, 122, 130. In another example, not show, the fluid path may form a serpentine shape where the sections extend generally axially along the length of body 82 rather than laterally as shown in FIG. 6. In another example, a flow restrictor 80 may comprise a fluid path 94 having both lateral/transverse sections and axial sections. Other variations on serpentine patterns may be used.

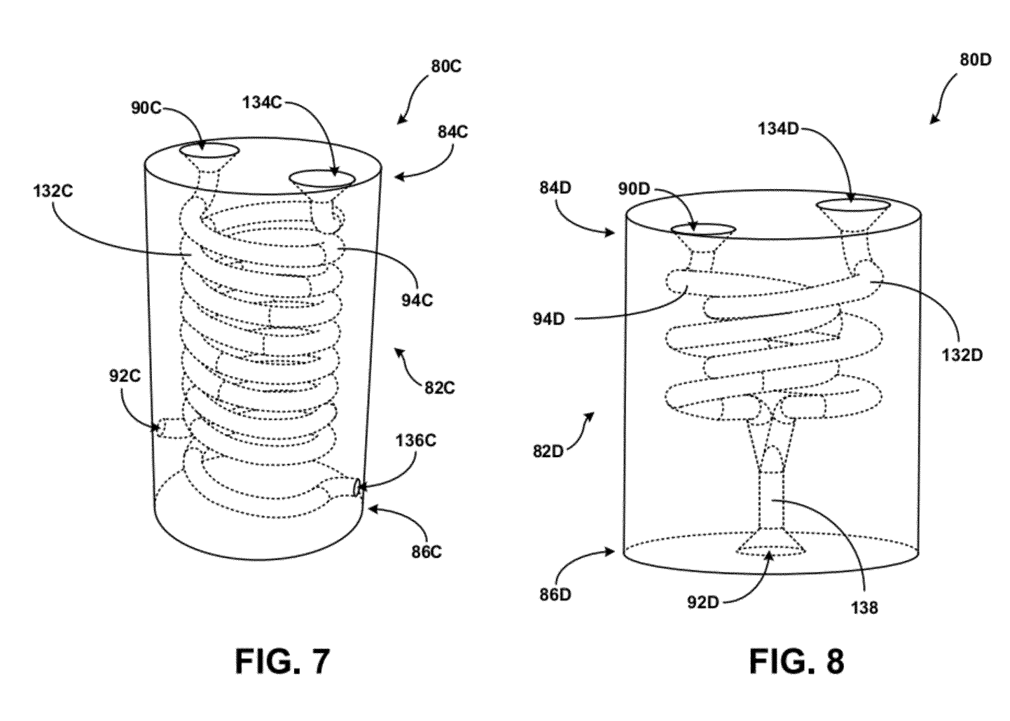

As shown in FIGS. 5 and 6, an example flow restrictor 80 may comprise a single fluid path 94, wherein the single fluid path 94 is in fluid communication with a single fluid lumen within a catheter, such as lumen 120 within catheter 14 (FIG. 6). In some examples, however, the flow restrictor may comprise a plurality of fluid paths. For example, as shown in FIG. 7, example flow restrictor 80C may comprise a first fluid path 94C extending from first fluid inlet 90C to first fluid outlet 92C and a second fluid path 132C, wherein second fluid path 132C extends from a second fluid inlet 134C to a second fluid outlet 136C. Like the first fluid path 94C, the second fluid path 132C may comprise any of several geometries that provide for a desired length of the second fluid path. In one example, both first fluid path 94C and second fluid path 132C comprise a generally helical shape, such as the generally concentric and axially offset helices of fluid paths 94C and 132C shown in FIG. 7.

In one example, not shown, first fluid path 94C may be in fluid communication with a first fluid lumen within catheter 14 and second fluid path 132C may be in fluid communication with a second fluid lumen within catheter 14. In such a configuration, a first therapeutic fluid can be delivered through the first fluid lumen and then through first fluid path 94C while a second therapeutic fluid can be delivered through the second fluid lumen and then through second fluid path 132C so that the first therapeutic fluid and the second therapeutic fluid can be delivered to the target site. In another example, both first fluid path 94C and second fluid path 132C may be in fluid communication with a common fluid lumen, such as lumen 120 of catheter 14, such that the same therapeutic fluid is being delivered to both fluid paths 94E and 132E. Multiple fluid paths 94E, 132E delivering the same therapeutic fluid may be used so that the therapeutic fluid is dispersed evenly at the target site.

In another example, not shown, a flow restrictor 80 may comprise a first fluid path and a second fluid path, wherein each fluid path is generally helical and the two fluid paths are generally concentric, but with one fluid path having a helical diameter that is smaller than a helical diameter of the second fluid path so that first fluid path radially fits within the second fluid path. In one example, it may be desired that the length of the first fluid path be generally the same as the length of the second fluid path. Because of this, and because the second fluid path has a larger helical diameter than the helical diameter of the first fluid path, the second fluid path may have a different pitch from a helical pitch of the first fluid path. Specifically, the second fluid path may have a larger helical pitch so that there are fewer turns of the second fluid path to compensate for the larger helical diameter.

FIG. 8 shows another example flow restrictor 80D wherein a body 82D defines a first fluid path 94D fed by a first fluid inlet 90D and a second fluid path 132D fed by a second fluid inlet 134D. Fluid paths 94D and 132D join together at a junction portion 138 which exits body 82D at a common fluid outlet 92D. In one example, shown in FIG. 8, each fluid path 94D, 132D is generally helical in shape before joining at junction portion 138. As described above, the overall length of each fluid path 94D, 132D and junction portion 138 may be selected by modifying the geometry of fluid paths 94D, 132D, 138, such as by selecting a helical diameter, a pitch length, and a number of helical turns of each fluid path 94D, 132D.

In one example, first fluid path 90D may be in fluid communication with a first lumen within catheter 14 while second fluid path 132D may be in fluid communication with a second lumen within catheter 14. This allows a first therapeutic fluid to be delivered to first fluid path 94D and a different, second therapeutic fluid to be delivered to the second fluid path 132D. The first and second therapeutic fluids are then joined in junction portion 138 where they are mixed to form a mixture of the first therapeutic fluid and the second therapeutic fluid.

In one example, first fluid path 94D and second fluid path 132D are substantially similar, e.g. having substantially the same geometry, for example the generally helical shape shown in FIG. 8, with substantially the same fluid path width (such as the same diameter if fluid paths 94D, 132D have a generally circular or elliptical cross section), substantially the same fluid path length, and substantially the same surface treatment, such that the flow rate of a first therapeutic fluid flowing from first path 94D into junction portion 138 is substantially the same as the flow rate of a second therapeutic fluid flowing from second fluid path 132D into junction portion 138. In such a case, the ratio of the first therapeutic fluid and the second therapeutic fluid exiting flow restrictor/mixer 80D will be substantially 1:1. In another example, first fluid path 94D and second fluid path 132D may be different in one or more respects, e.g., with a different geometry, a different fluid path width, a different fluid path length, and/or a different surface treatment, so that the flow rate of a first therapeutic agent flowing from first fluid path 94D into junction portion 138 is different from the flow rate of a second therapeutic agent flow from second fluid path 132D into junction portion 138. In such a case, the ratio between the first therapeutic fluid and the second therapeutic fluid may be adjusted by modifying the characteristics of each fluid path 94D, 132D to modify the relative flow rates within each fluid path 94D, 132D. A flow restrictor/mixer may be located at the end of a single catheter or at a junction between two or more catheters, or between a catheter extension and one or more catheters that join to deliver two or more therapeutic fluids to a common treatment location.

In one example, junction portion 138 is configured so that the first therapeutic fluid and the second therapeutic fluid are substantially evenly mixed before the combined therapeutic fluid exits from fluid outlet 92D. Flow restrictor/mixer 80D may be useful for therapeutic fluids that are desired to be delivered together, e.g., wherein the therapeutic fluids provide a combined therapeutic effect, but wherein it is undesirable for the two therapeutic agents to mix earlier, such as when a first of the therapeutic agents causes another therapeutic agent to become inactive over time, or when the combined therapeutic effect diminishes or deactivates over time. A flow restrictor/mixer may contain additional features (not shown) that promote the mixing of a first therapeutic fluid and a second therapeutic fluid, such as surface features within the fluid path that create sufficient viscosity to mix the first therapeutic fluid and the second therapeutic fluid.

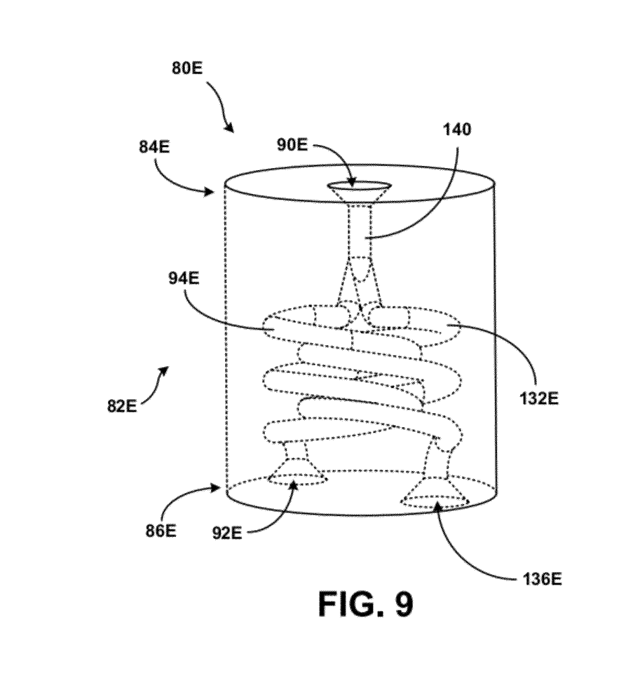

FIG. 9 shows an example flow restrictor 80E wherein a body 82E defines a common feed portion 140 that, within body 82E, splits into a first fluid path 94E and a second fluid path 132E. A common fluid inlet 90E feeds into feed portion 140, while first fluid path 94E exits body 82E at a first fluid outlet 92E and second fluid path 132E exits body 82E at a second fluid outlet 136E. In one example, fluid inlet 90E is in fluid communication with a lumen of a catheter, such as catheter 14, so that a therapeutic fluid that is delivered through the lumen is fed into feed portion 140 and split into a plurality of fluid paths 94E and 132E where the therapeutic fluid exits flow restrictor 80E at fluid outlets 92E and 126E.

Flow restrictor/splitter 80E allows a common therapeutic fluid to be delivered to multiple positions within a target tissue. A flow restrictor/splitter may be located at the end of a single catheter or at a junction between two or more catheters, or between a catheter extension and one or more catheters that deliver the therapeutic agent to multiple treatment locations. Flow restrictor/splitter 80E may also allow a larger total flow rate of therapeutic fluid to be delivered to the target tissue compared to a single fluid path while still allowing for a relatively high fluid velocity exiting at fluid outlets 92E, 126E. As described above, a small fluid path width at fluid outlet 92E, 126E, such as between about 1 micrometer and about 20 micrometers, and a relatively high fluid velocity at fluid outlet, such as between about 1 millimeters per second and about 20 millimeters per second, may prevent or decrease buildup of tissue, such as via cell migration into the fluid path, that may result in occlusion of fluid paths 94E, 132E. In some examples, a particular change in pressure between fluid inlet 90 and fluid outlet 92 may be sufficient to prevent or decrease tissue buildup, such as a change in pressure (ΔP) of about 50 kilopascals (about 7.25 pounds per square inch (PSI)) and about 400 kilopascals (about 58 PSI), such as between about 135 kilopascals (about 19.5 PSI) and about 250 kilopascals (about 36.3 PSI).

As described above, flow restrictor 80 may be very small, e.g., with a length of between about 0.25 millimeters and about 5 millimeters and a width (e.g., a diameter) of between about 0.25 millimeters and about 5 millimeters. Similarly, in some examples flow restrictor 80 may define a fluid path 94 that has a width (e.g. a diameter) of between about 1 micrometer and about 20 micrometers so that flow restrictor 80 can provide for a flow rate of therapeutic fluid of between about 0.25 microliters per hour and about 50 microliters per hour.

Traditional methods of forming fluid restrictors, such as molding, generally cannot produce the shapes of fluid path 94 described above on this small of a scale to produce such a small flow rate. Prior attempts at producing flow restrictors capable of providing flow rates on the order of 0.25 microliters per hour to 50 microliters per hour used conventional microfluidic devices that are generally made through lithography processing so that the fluid restrictors were bound to a two-dimensional or pseudo-two-dimensional geometry that may be undesirable for implantation in certain target tissues, such as within the brain 16 or spinal cord 30. Moreover, lithography processing often involves microfluidic devices made from glass or other materials that are brittle and neither biostable nor biocompatible within a patient 6.

Multi-photon polymerization (MPP) provides a means for microfabrication of flow restrictor 80 that is capable of producing the complex, three-dimensional geometries of fluid paths, such as the geometries of fluid paths 94A, 94B, 94C, 94D, 94E, 132C, 132D, 132E, 138, 140 described above with respect to FIGS. 5-9, at the desired size of fluid path 94 (e.g., with a width of fluid path of less than about 20 micrometers). In some examples, MPP comprises the use of a resin, such as a resin comprising a plurality of monomer molecules that react to form a solid polymer and a photoinitiator. The photoinitiator is activated by substantially simultaneous absorption (e.g. absorption within the same quantum event) of two or more photons within a small volume that induces reactions between the photoinitator and the monomer molecules to form a hardened polymer. The photoinitiation and resulting material hardening occurs within well defined and highly localized volume, referred to herein as a focal volume or a volumetric pixel (a “voxel”). In some examples, a laser capable of generating short laser pulses, such as femtosecond pulses, which can be focused into a desired focal volume in order to provide for MPP within the focal volume.

In one example, MPP comprises two-photon polymerization (2PP) wherein a photoinitiator in the resin is activated by substantially simultaneous absorption of two photons within a small volume. In one example, a laser configured to provide for substantially simultaneous delivery of two or more photons to a particular focal volume in order to initiate MPP. In one example, the laser may be capable of providing femtosecond laser pulses provides the light that provide the photons for 2PP. In one example, the laser is capable of producing pulses having a pulse width of between about 50 femtoseconds and about 100 femtoseconds, for example about 60 femtoseconds, at a frequency of between about 75 megahertz and about 100 megahertz, for example around 94 megahertz. The laser may be able to produce a peak power of about 450 milliwatts per pulse, and is capable of producing light having a wavelength of between about 750 nanometers and about 825 nanometers, such as a wavelength of about 780 nanometers. The femtosecond pulses are focused into a focal volume, such as via a high numerical aperture microscope objective lens. In one example, the laser is focused into a focal volume using an achromatic microscope objective.

Substantially simultaneous absorption of two photons within the same focal volume within a resin results in photoinitiation of a curing reaction between a photoinitiator and monomers within the resin. Uncured resin, such as resin within a lumen of a fluid path, may simply be washed away after the MPP process is complete. The curing results in a small volumetric pixel, or “voxel” of cured polymer having a known feature size FS_{V}, generally at the location of absorption. Further description of multi-proton polymerization, such as two-proton polymerization, may be found in Roger J. Narayan et al., “Medical prototyping using two photon polymerization,” Materials Today, Vol. 13, No. 12, December 2010, pp. 42-48, and Shaun D. Gittard et al., “Fabrication of Polymer Microneedles Using a Two-Photon Polymerization and Micromolding Process,” J. of Diabetes Sci. & Tech., Vol. 3, Issue 2, March 2009, pp. 304-311, the disclosures of which are incorporated in their entirety as if reproduced herein.

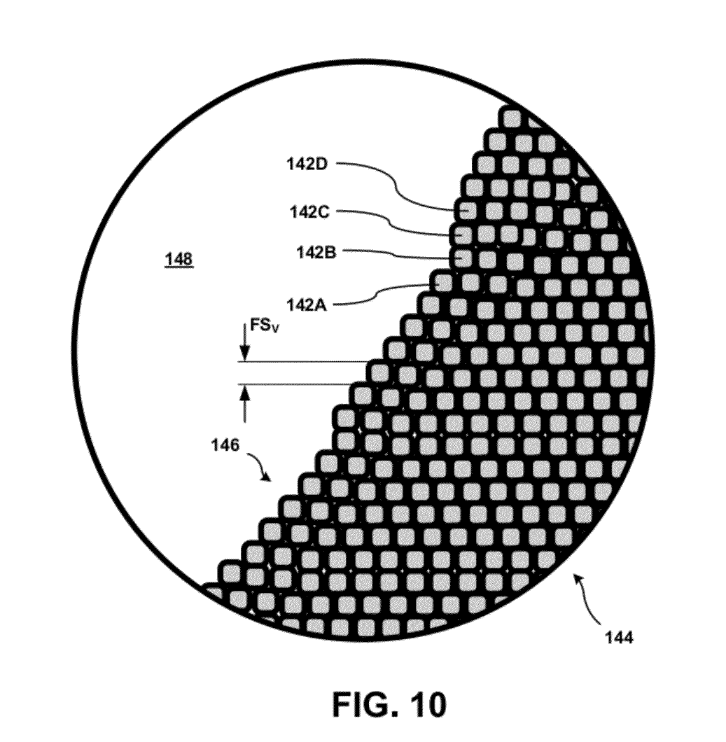

FIG. 10 is a conceptual cross-sectional view of a plurality of voxels 142A, 142B, 142C, 142D (collectively referred to herein as “voxel 142” or “voxels 142”) that have been cured by a focused laser, with each voxel 142 having a feature size FS_{V}. The minimum feature size of a voxel may be dependent on several parameters, including exposure time to the laser, laser power, the numerical aperture of the objective lens, sensitivity of the resin, and the voxel-to-voxel distance. In one example, 2PP may be capable of producing voxels.

Each voxel 142 may be cured using MPP, such as 2PP, and a bursts or bursts from a laser configured to activate a photoinitiator via multi-photon absorption. After one voxel, such as voxel 142A, is cured, the focal volume of the laser may be moved to an adjacent location to form another voxel, such as a voxel 142B adjacent to the first voxel 142B. The process may be repeated (e.g., with third voxel 142C, fourth voxel 142D, and so on) until a desired geometry is formed from the plurality of individual voxels. In one example, the scanning path of focal volumes is moved along a first two-dimensional plane, e.g., along an X-Y plane that is a single voxel thick, to build the first plane, followed by translating the focal volume up, e.g., in the Z axis, and building a second two-dimensional plane on top of the first two-dimensional plane. The geometry of a flow restrictor 80 may be created by either “contour scanning” or “raster scanning” “Contour scanning,” as it is used herein, refers to solidifying only the contour of flow restrictor 80, for example the outer surfaces and the shape of fluid path 94, where the remaining bulk of flow restrictor 80 is cured in a post-MPP curing step. The post-MPP curing may comprise exposing the resin within the contoured shape to a light source that activates and cures the remaining resin. The phrase “raster scanning,” as it is used here, refers to using the MPP laser to scan and solidify the entire volume of flow restrictor 80, including the bulk, voxel by voxel. After the MPP process is complete, left over resin may be washed away leaving behind a completed flow restrictor 80.

FIG. 10 shows a portion of a two-dimensional plane 144 that has been built to form an inner diameter 146 of a fluid path 148, such as fluid paths 94, 132, 138, and 140. The MPP process may continue to build plane 144 so that the entirety of inner diameter 146 is formed and, in the case of raster scanning, so that the entire cross section of a flow restrictor at the plane 144 is completed. Because the feature size FS_{V} of each voxel 142 is small, e.g., less than about 100 nanometers, such as between about 75 nanometers and about 100 nanometers, MPP to form voxels 142 may be used to form seemingly continuous surfaces down to the order of single micrometers, e.g., between about 1 micrometer (about 1000 nanometers) and about 20 micrometers (20000 nanometers).

As noted above, the resin that is cured via MPP comprises a photoinitiator that is activated by the substantially simultaneous absorption of two or more photons (e.g. absorption in the same quantum event) and a monomer that reacts with the activated photoinitiator and polymerizes to cure into a polymer. Examples of the photoinitiator include, but are not limited to, a-hydroxyketone photoinitiators, bis-acyl phosphine oxide (BAPO) photoinitiators, α-aminoketone photoinitiators, and azobisisobutronitrile (AIBN). In one example, a photoinitiator sold under the IRGACURE trade name by Ciba Specialty Chemicals (Basel, Switzerland) may be used as the photoinitiator, such as IRGACURE 369 having an absorption peak at a wavelength of about 320 nanometers.

Examples of the monomer include, but are not limited to biostable/biocompatible light-curable polymers such as acrylate-based polymers, organically-modified ceramic materials, zirconium sol-gels, titanium-containing hybrid materials, methacrylates, and light-curable chitosans. In one example, an inorganic-organic hybrid polymer, such as the hybrid polymers sold under the trade name ORMOCER by Fraunhofer ISC (Würzburg, Germany), which may comprise an organic network (e.g., a hydrocarbon backbone) with glass/ceramic functional groups (e.g., silicate, titanate, and zirconate functional groups) and/or silicone functional groups (e.g., silanes or silyl functional groups). Physical properties of the ORMOCER polymer may be modified based on the functional group makeup, e.g., the surface energy may be modified by selecting appropriate silicone functional groups while hardness, chemical stability, and thermal stability may be modified by selecting appropriate glass/ceramic functional groups.

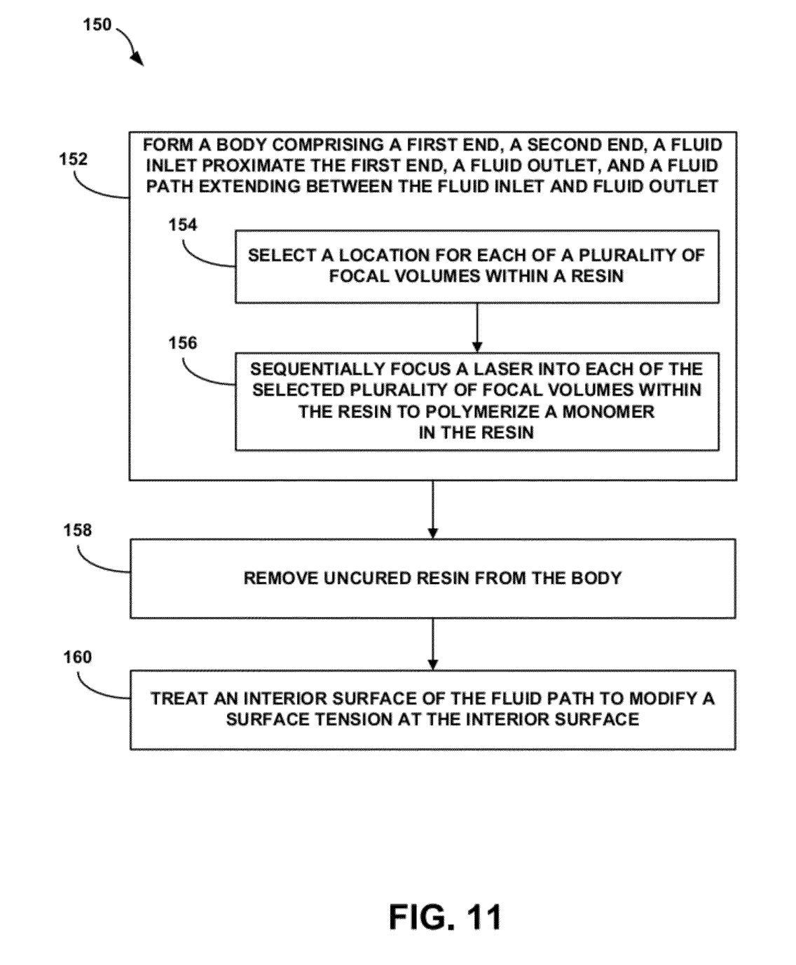

FIG. 11 is a flow chart illustrating an example method 150 of making a flow restrictor for a medical device, such as flow restrictor 80 shown in FIGS. 1 and 4. The example method 150 comprises forming a body 82 (152) comprising a first end 84, a second end 86, a fluid inlet 90 proximate first end 84, a fluid outlet 92, and a fluid path 94 extending between fluid inlet 90 and fluid outlet 92. Forming at least a portion body 82 (152) comprises selecting a location for each of a plurality of focal volumes within a resin (154), wherein the resin comprises a monomer and a photoinitiator sensitive to light having a wavelength range. The photoinitiator is configured to initiate polymerization of the monomer within one of the plurality focal volumes when two or more photons of light within the wavelength range are substantially simultaneously absorbed by the photoinitiator within the one of the plurality of focal volumes, e.g., when the photoinitiator absorbs two or more photons within the same quantum event. The plurality of selected focal volumes form a shape of the portion of body 82, such as the shape of fluid path 94 or the entirety of body 82.

After selecting the location of the plurality of focal volumes (154), the example method 150 further comprises sequentially focusing a laser into each of the plurality of focal volumes within the resin to polymerize the monomer and form the portion of body 82 (156). The laser may be configured to provide for multi-photon absorption, such as two-photon absorption, at the wavelength range within each of the plurality of selected focal volumes. In one example, focusing the laser into each of the plurality of focal volumes to polymerize the monomer (156) may comprise polymerizing the monomer to form a voxel 142 having a feature size FS_{V} that is approximately equal to a size of the focal volume of the laser. In one example, method 150 further comprises removing uncured resin from body 82 (158). Removing the uncured resin (158) may comprise washing the uncured resin away from the cured polymer that forms body 82. In one example, an organic solvent may be used to wash out the uncured resin.

Method 150 may further comprise treating an interior surface of fluid path 94, e.g., an interior diameter of fluid path 94 along the length of fluid path 94, to modify a surface tension at the interior surface (160). Treating the interior surface of fluid path 94 (160) may comprise at least one of plasma treating the interior surface, chemically treating the interior surface, coating the interior surface with a surface treatment, fluorinating the interior surface, and oxidizing the interior surface. Treating the interior surface allows the surface tension at the interior surface of fluid path 94 to be selected, which in turn may control the contact angle between the therapeutic fluid being delivered through fluid path 94 and the solid material of flow restrictor 80. The contact angle is known to affect flow behavior within fluid path 94, such as the shear rate of fluid flowing through fluid path 94 or the formation of laminar flow. For example, for some therapeutic fluids comprising a protein therapeutic agent, the protein may become damaged or denatured when subjected to excessive shear forces. In one example, the shear forces may be reduced by fluorinating the interior surface, such as via treatment with a fluorine plasma, which increases the contact angle. Conversely, if it is desired to achieve greater shear forces, e.g., to provide a slower flow rate through fluid path 94, then the interior surface may be oxidized, which decreases the contact angle.

Various examples have been described. These and other examples are within the scope of the following claims.

1. A method comprising applying a multi-photon polymerization (MPP) process to a material to define a fluid flow restrictor for a medical device.

2. The method of claim 1, wherein applying the MPP process comprises:
selecting a location for each of a plurality of focal volumes within a resin, the resin comprising a monomer and a photoinitiator sensitive to light having a wavelength range, wherein the photoinitiator is configured to initiate polymerization of the monomer within one of the plurality of focal volumes when two or more photons of light having the wavelength range are absorbed by the photoinitiator within the one of the plurality of focal volumes, wherein the plurality of selected focal volumes form a shape of a portion of a body defining the fluid flow restrictor; and
sequentially focusing a laser into each of the plurality of selected focal volumes within the resin to polymerize the monomer and form the portion of the body, wherein the laser is configured to provide for multi-photon absorption at the wavelength range within each of the plurality of selected focal volumes.

3. The method of claim 2, wherein the body defining the fluid flow restrictor comprises a fluid path between a fluid inlet and a fluid outlet.

4. The method of claim 3, wherein the fluid path has a three-dimensional geometry.

5. The method of claim 3, wherein the fluid path has a geometry comprising at least one of a generally cylindrically helical shape, a generally conically helical shape, or a serpentine shape, wherein the geometry is selected to provide the predetermined length of the fluid path.

6. The method of claim 3, further comprising treating an interior surface of the fluid path to modify surface tension at the interior surface.

7. The method of claim 6, wherein treating the interior surface comprises at least one of plasma treating the interior surface, chemically treating the interior surface, coating the interior surface with a surface treatment, fluorinating the interior surface, and oxidizing the interior surface.

8. The method of claim 2, wherein the body defining the fluid flow restrictor comprises an attachment structure configured for attaching the body to a catheter.

9. The method of claim 8, further comprising attaching the body to the catheter via the attachment structure.

10. The method of claim 2, wherein each of the plurality of focal volumes has a size of less than about 100 nanometers.

11. The method of claim 2, wherein the laser is configured to provide laser pulses.

12. The method of claim 11, wherein the laser pulses have a pulse width between about 50 femtoseconds and about 100 femtoseconds and a frequency of between about 75 megahertz and about 100 megahertz.

13. The method of claim 2, wherein focusing the laser into each of the plurality of focal volumes comprises focusing the laser using a microscope objective.

14. The method of claim 2, wherein each of the plurality of focal volumes has a feature size of less than about 100 nanometers.

15. The method of claim 2, wherein the plurality of selected focal volumes form a shape of the entire body, and wherein sequentially focusing the laser into each of the plurality of selected focal volumes forms the entire body.

16. The method of claim 2, wherein selecting a location for each of a plurality of focal volumes comprises selecting a plurality of focal volumes to form a contour of a fluid path and sequentially focusing the laser into each of the plurality of selected focal volumes comprises focusing the laser into each of the selected plurality of focal volumes that form the contour.

17. The method of claim 2, wherein the body comprises a first fluid path extending between a first fluid inlet and a first fluid outlet and a second fluid path extending between a second fluid inlet and a second fluid outlet.

18. The method of claim 2, wherein the body comprises a first fluid path extending from a first fluid inlet and a second fluid path extending from a second fluid inlet, wherein the first fluid path and second fluid path converge to form a junction portion extending to the fluid outlet.

19. The method of claim 2, wherein the body comprises a fluid inlet portion extending from a fluid inlet, wherein the fluid inlet portion splits into a first fluid path extending to a first fluid outlet and a second fluid path extending to a second fluid outlet.

20. A flow restrictor for a medical device, the flow restrictor comprising:
a body having a first end, a second end, a fluid inlet proximate the first end, a fluid outlet, and a fluid path between the fluid inlet and the fluid outlet;
wherein the body is made from a photocrosslinkable polymer that is formed by multi-photon polymerization of the polymer.

21. The flow restrictor of claim 20, wherein the body further comprises an attachment structure configured for attaching the body to a catheter.

22. The flow restrictor of claim 20, wherein the fluid path has a width and a length selected to provide a predetermined flow rate of a therapeutic agent through the body.

23. The flow restrictor of claim 20, wherein a cross-sectional width of the fluid path is between about 1 micrometer and about 20 micrometers.

24. The flow restrictor of claim 20, wherein a cross-sectional width of the fluid path is selected to provide a local fluid velocity that reduces or eliminates occlusion of the fluid path.

25. The flow restrictor of claim 20, wherein the fluid path has a substantially circular cross-sectional shape, wherein an inner diameter of the fluid path is between about 1 micrometer and about 20 micrometers.

26. The flow restrictor of claim 20, wherein the fluid path has a geometry comprising at least one of a substantially cylindrically helical shape, a generally conically helical shape, a generally serpentine shape, wherein the geometry is selected to provide the predetermined length of the fluid path.

27. The flow restrictor of claim 20, wherein the photocrosslinkable polymer comprises an inorganic-organic hybrid polymer.

28. The flow restrictor of claim 20, wherein the body further comprises a surface treatment on an interior surface of the fluid path to modify surface tension at the interior surface.

29. The flow restrictor of claim 28, wherein the surface treatment comprises at least one of a plasma treatment, a chemical treatment, a coating, a fluorinating treatment, and an oxidizing treatment.

30. The flow restrictor of claim 20, wherein the body comprises a first fluid path extending between a first fluid inlet and a first fluid outlet and a second fluid path extending between a second fluid inlet and a second fluid outlet.

31. The flow restrictor of claim 20, wherein the body comprises a first fluid path extending from a first fluid inlet and a second fluid path extending from a second fluid inlet, wherein the first fluid path and second fluid path converge to form a junction portion extending to the fluid outlet.

32. The flow restrictor of claim 20, wherein the body comprises a fluid inlet portion extending from a fluid inlet, wherein the fluid inlet portion splits into a first fluid path extending to a first fluid outlet and a second fluid path extending to a second fluid outlet.

33. A system comprising:
a fluid delivery device;
a catheter comprising a proximal end coupled to the fluid delivery device, a distal end implantable proximate a target tissue, and a lumen extending from the proximal end to the distal end;
a flow restrictor coupled to the distal end of the catheter, the flow restrictor comprising a body having a first end, a second end, a fluid inlet proximate the first end in fluid communication with the catheter lumen, a fluid outlet, and a fluid path between the fluid inlet and the fluid outlet;
wherein the flow restrictor body is made from a photocrosslinkable polymer that is formed by multi-photon polymerization of the polymer.

34. The system of claim 33, wherein the flow restrictor body further comprises an attachment structure configured for attaching the body to the catheter.

35. The system of claim 33, wherein the fluid path has a width and a length selected to provide a predetermined flow rate of a therapeutic agent through the body.

36. The system of claim 33, wherein a cross-sectional width of the fluid path is between about 1 micrometer and about 20 micrometers.

37. The system of claim 33, wherein the fluid path has a substantially circular cross-sectional shape, wherein an inner diameter of the fluid path is between about 1 micrometer and about 20 micrometers.

38. The system of claim 33, wherein the fluid path has a geometry comprising at least one of a substantially cylindrically helical shape, a generally conically helical shape, a generally serpentine shape, wherein the geometry is selected to provide the predetermined length of the fluid path.

39. The system of claim 33, wherein the photocrosslinkable polymer comprises an inorganic-organic hybrid polymer.

40. The system of claim 33, wherein the flow restrictor body further comprises a surface treatment on an interior surface of the fluid path to modify surface tension at the interior surface.

41. The system of claim 40, wherein the surface treatment comprises at least one of a plasma treatment, a chemical treatment, a coating, a fluorinating treatment, and an oxidizing treatment.

42. The system of claim 33, wherein the flow restrictor body comprises a first fluid path extending between a first fluid inlet and a second fluid inlet and a second fluid path extending between a second fluid inlet and a second fluid outlet.

43. The system of claim 33, wherein the flow restrictor body comprises a first fluid path extending from a first fluid inlet and a second fluid path extending from a second fluid inlet, wherein the first fluid path and second fluid path converge to form a junction portion extending to the fluid outlet.

44. The system of claim 33, wherein the flow restrictor body comprises a fluid inlet portion extending from a fluid inlet, wherein the fluid inlet portion splits into a first fluid path extending to a first fluid outlet and a second fluid path extending to a second fluid outlet.
